(19) United States
(12) Patent Application Publication
(10) Pub. No.: US 2024/0157170 A1
(43) Pub. Date: May 16, 2024

(54) MULTI-LAYER PORTABLE THERAPEUTIC INFRARED HEATING SYSTEM AND DEVICE

(71) Applicant: HigherDOSE LLC, Brooklyn, NY (US)

(72) Inventors: Mary Kathleen Kaps, Miami Beach, FL (US); Lauren Berlingeri, Putnam Valley, NY (US); Dong Hak Lee, Kwangju City Kyoungkido (KR)

(21) Appl. No.: 18/350,356

(22) PCT Filed: Nov. 11, 2022
(86) PCT No.: PCT/US22/49685

§ 371 Date: Jul. 11, 2023

Related U.S. Application Data
(60) Provisional application No. 63/279,828, filed on Nov. 16, 2021.

(51) Int. Cl. A61N 5/06; A61F 7/00; A61L 31/02; A61L 31/14; A61N 2/00

(52) U.S. Cl. CPC A61N 5/0625; A61F 7/0097; A61L 31/028; A61L 31/14; A61N 2/002; A61N 2/004; A61F 2007/0071; A61F 2007/0088; A61F 2007/0095; A61N 1/16; A61N 2005/066

(57) ABSTRACT

A multi-layer portable therapeutic infrared heating system comprises a heating wire layer configured to generate far-infrared radiation (FIR) when the multi-layer portable therapeutic infrared heating system is connected with an alternating current power supply; two charcoal layers positioned on either side of the heating wire layer and configured to absorb extremely low frequency (ELF) radiation associated with the FIR; grounding mechanism configured to transfer the ELF radiation absorbed by the two charcoal layers to a pin and ground the multi-layer portable therapeutic infrared heating system; and a multi-layer mixture layer configured to at least additionally reduce the ELF radiation.

### CROSS REFERENCE TO RELATED APPLICATIONS

This application is a national phase application of international application No. PCT/US22/49685, filed on Nov. 11, 2022, which claims priority to U.S. Provisional Patent Application No. 63/279,828 filed on Nov. 16, 2021, entitled “MULTI-LAYER PORTABLE THERAPEUTIC INFRARED HEATING SYSTEM AND DEVICE,” the contents of which are incorporated by reference herein in their entirety.

### FIELD OF TECHNOLOGY

The present disclosure generally relates to an electronic system and device for heating the body of a user using far-infrared radiation technology, and more particularly relates to a multi-layer portable therapeutic infrared heating system and device for achieving various therapeutic effects.

### BACKGROUND

Conventional heat therapy may be hazardous, uncomfortable, unhygienic, inefficient, ineffective and inconvenient for travel purposes. For example, conventional saunas may create exposure to extremely high temperatures causing heat stress which may have detrimental effects on a user's health, especially for people with heart challenges or needing to sustain blood pressure.

Accordingly, there is a need for a safe, effective and portable therapeutic infrared heating system and device for achieving various therapeutic effects.

### SUMMARY

The present disclosure provides a multi-layer portable therapeutic infrared heating system and device for boosting immunity, boosting blood flow and circulation, and boosting mood of a user.

In one aspect, the present disclosure generally relates to a multi-layer portable therapeutic infrared heating system, comprising: a heating wire layer configured to generate far-infrared radiation (FIR) when the multi-layer portable therapeutic infrared heating system is connected with an alternating current power supply; two charcoal layers positioned on either side of the heating wire layer and configured to absorb extremely low frequency (ELF) radiation associated with the FIR; grounding mechanism configured to transfer the ELF radiation absorbed by the two charcoal layers to a pin and ground the multi-layer portable therapeutic infrared heating system; and a multi-layer mixture layer configured to at least additionally reduce the ELF radiation.

In one embodiment, the heating wire layer of the multi-layer portable therapeutic infrared heating system may include silicone Teflon wires. Further, the system may be configured to maintain a level of the ELF below 5 mV and low frequency electromagnetic field (EMF) below 2 milligauss. In another embodiment, the multi-layer mixture layer may include a selected amount of one or more of amethyst gravel, yellow mud ceramic ball, charcoal ceramic ball, white ceramic ball, and tourmaline ceramic ball. In one aspect, the system may include a grid to hold stones and materials of the multi-layer mixture layer together. The system may also include a medical magnet layer including multiple medical magnets for activating a blood circulation of a user of the system. In certain embodiments, the system may include a yellow mud charcoal bonding fabric layer configured to promote a generation of the FIR. Moreover, the system may include at least one charcoal layer configured to ground the multi-layer portable therapeutic infrared heating system and reduce the ELF radiation. The system may additionally include multiple volatile organic compounds (VOC) free polyurethane leather (PU) layers or portions for water-proof, fire-proof and melt-proof purposes.

In yet another embodiment, the system may include at least one electronic temperature sensor for monitoring and detecting an internal temperature of the multi-layer portable therapeutic infrared heating system and regulating a heating temperature of the multi-layer portable therapeutic infrared heating system in accordance with a selected temperature level.

A safety unit may also be included and configured to disconnect the heating wire layer when the at least one electronic temperature sensor detects that the internal temperature of the multi-layer portable therapeutic infrared heating system exceeds 80° C. In one aspect, the safety unit may be configured to connect to the heating wire layer when the internal temperature of the multi-layer portable therapeutic infrared heating system detected by the at least one electronic temperature sensor returns to a normal range.

In another embodiment, the system may include silver fiber fabric and copper mesh fabric portions or layers to additionally reduce the ELF radiation. The grounding mechanism of the system may comprise at least two grounding lead wires configured to connect the two charcoal layers with a grounding pin. The heating wire layer of the system may include wires made of a material selected to reduce or shield harmful electric and magnetic fields generated by the heating wire layer. The multi-layer mixture layer may further be configured to promote higher FIR generation, heating distribution and therapeutic benefits.

In accordance with additional aspects of the present disclosure, a multi-layer portable therapeutic infrared heating blanket may comprise a heating wire layer configured to generate FIR when the multi-layer portable therapeutic infrared heating blanket is connected with an alternating current power supply; two charcoal layers positioned on either side of the heating wire layer and configured to absorb ELF radiation associated with the FIR; grounding mechanism configured to transfer the ELF radiation absorbed by the two charcoal layers to a pin and ground the multi-layer portable therapeutic infrared heating blanket; and a multi-layer mixture layer configured to at least additionally reduce the ELF radiation.

In one embodiment, the heating wire layer of the multi-layer portable therapeutic infrared heating blanket may include silicone Teflon wires. The multi-layer portable therapeutic infrared heating blanket may be configured to maintain a level of the ELF below 5 mV and low frequency EMF below 2 milligauss. The multi-layer mixture layer may include a selected amount of one or more of amethyst gravel, yellow mud ceramic ball, charcoal ceramic ball, white ceramic ball, and tourmaline ceramic ball. A grid may be included to hold stones and materials of the multi-layer mixture layer together. In addition, the multi-layer portable therapeutic infrared heating blanket may include a medical magnet layer having multiple medical magnets for activating a blood circulation of a user of the multi-layer portable therapeutic infrared heating blanket.

In certain embodiments, a yellow mud charcoal bonding fabric layer may be included and configured to promote a generation of the FIR. At least one charcoal layer may be included and configured to ground the multi-layer portable therapeutic infrared heating blanket and reduce the ELF radiation. Further, multiple VOC free PU layers or portions may be included for water-proof, fire-proof and melt-proof purposes.

In another embodiment, at least one electronic temperature sensor may be included for monitoring and detecting an internal temperature of the multi-layer portable therapeutic infrared heating blanket and regulating a heating temperature of the multi-layer portable therapeutic infrared heating blanket in accordance with a selected temperature level.

In yet another embodiment, a safety unit may be included and configured to disconnect the heating wire layer when the at least one electronic temperature sensor detects that the internal temperature of the multi-layer portable therapeutic infrared heating blanket exceeds 80° C. In one aspect, the safety unit may be configured to connect to the heating wire layer when the internal temperature of the multi-layer portable therapeutic infrared heating blanket detected by the at least one electronic temperature sensor returns to a normal range.

In yet another embodiment, silver fiber fabric and copper mesh fabric portions or layers may be included to additionally reduce the ELF radiation. The grounding mechanism may comprise at least two grounding lead wires configured to connect the two charcoal layers with a grounding pin. The heating wire layer may include wires made of a material selected to reduce or shield harmful electric and magnetic fields generated by the heating wire layer. The multi-layer mixture layer may be further configured to promote higher FIR generation, heating distribution and therapeutic benefits.

In a preferred embodiment, the multi-layer portable therapeutic infrared heating blanket may comprise a zipper configured to open the multi-layer portable therapeutic infrared heating blanket from one side for easy access. In one aspect, the zipper may be configured to start at one corner of a bottom of the multi-layer portable therapeutic infrared heating blanket. Moreover, the zipper may be configured to close a portion of the multi-layer portable therapeutic infrared heating blanket on one side at a selected position.

The above simplified summary of example aspects serves to provide a basic understanding of the present disclosure. This summary is not an extensive overview of all contemplated aspects, and is intended to neither identify key or critical elements of all aspects nor delineate the scope of any or all aspects of the present disclosure. Its sole purpose is to present one or more aspects in a simplified form as a prelude to the more detailed description of the disclosure that follows. To the accomplishment of the foregoing, the one or more aspects of the present disclosure include the features described and exemplary pointed out in the claims.

### BRIEF DESCRIPTION OF THE DRAWINGS

The accompanying drawings, which are incorporated into and constitute a part of this specification, illustrate one or more example aspects of the present disclosure and, together with the detailed description, serve to explain their principles and implementations.

FIG. 1 illustrates a general electrical wiring diagram of a multi-layer portable therapeutic infrared heating system, according to an exemplary aspect of the present disclosure;

FIG. 2 illustrates various portions, components and layers of a multi-layer portable therapeutic infrared heating system, according to an exemplary aspect of the present disclosure;

FIG. 3(a) illustrates a conventional silicone heating wire;

FIG. 3(b) illustrates a silicone Teflon heating wire used in the multi-layer portable therapeutic infrared heating blanket of FIGS. 1 and 2, according to an exemplary aspect of the present disclosure;

FIG. 4 illustrates a multi-layer portable therapeutic infrared heating blanket in an open form, according to an exemplary aspect of the present disclosure; and

FIG. 5 illustrates a multi-layer portable therapeutic infrared heating blanket in a closed form, according to an exemplary aspect of the present disclosure.

### DETAILED DESCRIPTION

Various aspects of the present disclosure will be described with reference to the drawings, wherein like reference numerals are used to refer to like elements throughout. In the following description, for purposes of explanation, numerous specific details are set forth in order to promote a thorough understanding of one or more aspects of the present disclosure. It may be evident in some or all instances, however, that any aspects described below can be practiced without adopting the specific design details described below.

The system and device disclosed in the present disclosure may be configured to generate and deliver far-infrared rays or radiation (FIR) (λ=15 μm-1 mm) to the human body to achieve various therapeutic and health benefits. For a complete electromagnetic radiation spectrum, the infrared radiation (IR) band generally covers the wavelength range of 700 nm-1 mm, frequency range of 300 GHz-430 THz, and photon energy range of 12.4 meV-1.7 eV. In the IR radiation bands, only FIR transfers energy purely in the form of heat which can be perceived by the thermos-receptors in human skin as radiant heat. Specifically, FIR generally includes waves of energy that are invisible to the naked eye and penetrate the surface of the skin of a user to elevate the body's surface temperature to 107.6 Fahrenheit or 42° C. and above, while positively activating body systems and functions. The system and device disclosed in the present disclosure may be configured to utilize FIR to improve blood circulation and skin complexion. In one aspect, the disclosed system and device may be configured to expand capillaries which in turn stimulates increased blood flow, regeneration, circulation and oxygen. The disclosed system and device may also maintain and promote healthy lung function. For example, if sebaceous glands of a person are activated by the disclosed system and device, accumulated cosmetics in pores may be eliminated through the skin via sweat and oil glands, resulting in clearer skin.

In another example, the disclosed system and device may be configured to strengthen the cardiovascular system of a user by causing heart rate and cardiac output to increase, and diastolic blood pressure to decrease. Research has shown that FIR and the gentle tissue warming effect may improve the health of blood vessel cells of a user. Further, the disclosed system and device may be configured to detox and help lymphatic cleansing. For example, during the FIR treatment of clogged capillary vessels of a user, heat expands the capillaries and then initiates the start of a process to dissolve hidden toxins.

In yet another example, the disclosed system and device may be configured to promote the elimination of fats, chemicals and toxins from the blood: poisons, heavy metals—carcinogenic substances from food processing—lactic acid, free fatty acids, and subcutaneous fat associated with aging and fatigue—excess sodium associated with blood pressure—and uric acid. Where these toxins accumulate, blood circulation of a user may be blocked and cellular energy may be impaired. When FIR waves are applied to large water molecules and the water begins to vibrate. This vibration may reduce the ion bonds of the atoms that are holding together the molecules of water. As the vibration continues, breakdown of the water molecules occurs and encapsulated gases and other toxic materials may be released.

In addition, the disclosed system and device may be configured to achieve more rapid wound healing that may be independent of changes in blood flow and skin temperature. In one example, the FIR waves generated by the disclosed system and device may be configured to facilitate healing by stimulating a group of cells called fibroblasts to produce collagen which is a very important part of good wound healing and tissue repair. Furthermore, the disclosed system and device support cell growth, DNA syntheses, and protein synthesis that are all necessary during tissue repair and regeneration. In one embodiment, the disclosed system and device may be used to stimulate collagen production for healing burns, scar tissue and skin problems such as acne and skin breakouts.

Moreover, the disclosed system and device may be configured to generate and deliver FIR to relieve nervous tension and relax auto-neuro muscles, thereby facilitating a user's body to make the most of its intended healing abilities. In one embodiment, the FIR therapy of the present disclosure reduces soreness on nerve endings and muscle spasms, as muscle fibers are heated. As a result, rapid reduction of swelling, inflammation and associated muscular pain of e.g., back, shoulder and neck occurs. That is, the FIR therapy of the present disclosure soothes tension and stress in both body and mind of a user.

In yet another embodiment, the disclosed system and device may be configured to generate and deliver FIR to strengthen and support a user's immune system by supporting increased production of white blood cells (leukocytes) by the bone marrow and killer T-cells by the thymus. For example, FIR generated and delivered by the disclosed system and device may support in the destruction of malignant cells which cannot survive if the temperature of the cell becomes 107.6 Fahrenheit or 42° C. and above.

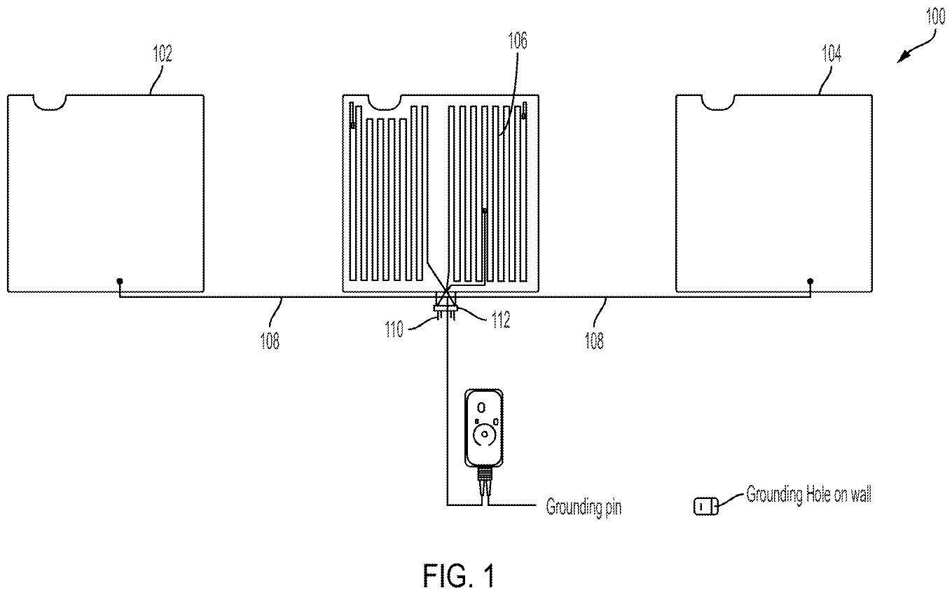

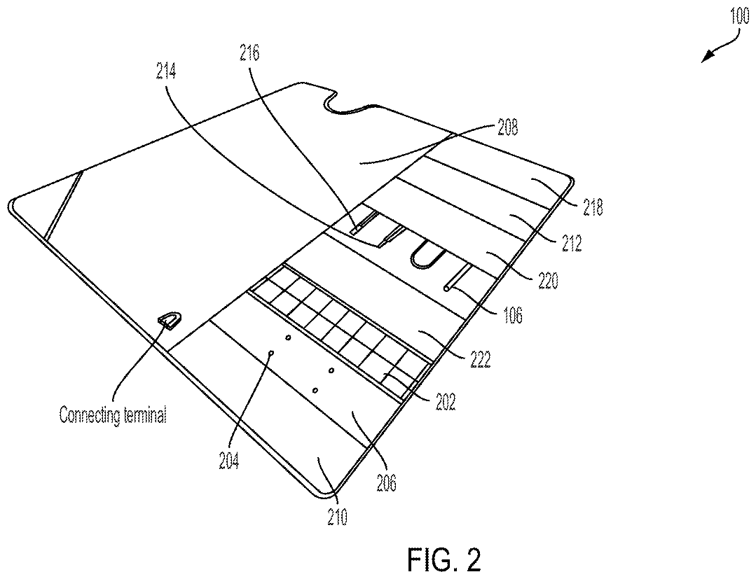

FIGS. 1 and 2 illustrate a multi-layer portable therapeutic heating system and device 100 configured to increase the core body temperature of a user (e.g., for drawing out metals and toxins through sweating of the user), in accordance with aspects of the present disclosure. In a preferred embodiment as shown in FIGS. 1 and 2, system 100 may be in the form of an infrared blanket including a unique combination of two charcoal layers 102 (e.g., on heating wires 106), 104 (e.g., beneath heating wires 106) positioned on either side of heating wires 106 for absorbing harmful extremely low frequency (ELF) waves or radiation. That is, charcoal layers 102, 104 may be respectively positioned on and beneath heating wires 106 when system 100 is connected with an external alternating current (AC) power supply and when heating wires 106 are emitting heat and ELF radiation on either side of the wires. Further, as shown in FIG. 1, at least two grounding lead wires 108 may be installed and used in system 100 for connecting the dual charcoal layers 102, 104 with a grounding pin 110 on the wall, thereby additionally reducing the harmful ELF radiation emitted from operating heating wires 106. Specifically, the dual charcoal layers 102, 104 of the present disclosure may be configured to absorb the maximum amount of ELF generated by the heating wires 106 and transfer it to a pin in a power terminal 112 in the blanket which functions as a medium to ground the blanket when it is connected to a wall outlet with grounding. In comparison to the international safety zone of 10 mV required for infrared and electronic sauna devices, system 100 of the present disclosure advantageously generates and delivers equal to or less than 5 mV ELF radiation. Among other features, the unique combination of dual charcoal layer design and grounding mechanism allows system 100 of the present disclosure to generate the maximum amount of FIR (approximately 92% FIR with a wavelength of 920 micrometer) while maintaining the ELF levels below 5 mV.

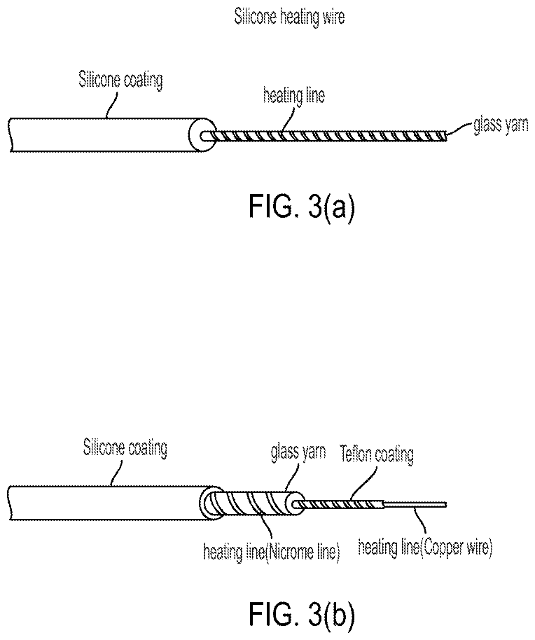

Referring to FIG. 3(a), a conventional heating wire may be made of glass yarn corn with heating lines wrapped around. Silicone coating may be applied to enclose the glass yarn on the outside and provide electrical insulation, heat resistance and cold resistance.

In accordance with aspects of the present disclosure, heating wires 106 may comprise silicone Teflon wires. A silicone Teflon wire may refer to a wire that is insulated with a mixture of silicone and Teflon, both exhibit great heat resistance. Having silicone as a part of the wire may provide the flexibility to bend the wires inside the blanket of system 100. As shown in FIG. 3(b), the silicone Teflon wires may have a multi-layer structure including a Copper line core as the heating line coated with a Teflon coating. A glass yarn may enclose the Teflon coated heating line and have additional heating line (Nicrome line) wrapped around. For example, Nichrome wire, when heated to certain temperatures (e.g., red-hot temperatures), may develop an outer layer of chromium oxide, which is thermodynamically stable in air, is mostly impervious to oxygen, and protects the heating element from further oxidation. Silicone coating may be applied on the outside of the heating wires of system 100 to provide electrical insulation, heat resistance and cold resistance. In one aspect, heating wires 106 may be configured to generate low frequency electromagnetic field (EMF) below 2 milligauss (MG) which is below the safety zone of 10 MG set by relevant regulations. In contrast, blankets that were tested and not using the heating wires 106 of the present disclosure generate EMF in the range of 60-70 MG which is harmful to a user.

It should be appreciated that any suitable wires may be used in system 100 to maximize a function of reducing or shielding harmful electric and magnetic fields generated by operating heating wires 106 and minimize the damage to a human body by such harmful electric and magnetic fields.

Referring to FIG. 2, in accordance with aspects of the present disclosure, system 100 may comprise a five-stone mixture portion or layer 202 to promote higher FIR generation, heating distribution and therapeutic benefits. In a preferred embodiment, layer 202 may include a selected amount of one or more of amethyst gravel, yellow mud ceramic ball, charcoal ceramic ball, white ceramic ball, and tourmaline ceramic ball. These stones or any suitable materials determined and selected for the five-stone mixture layer 202 may function to intensify or promote emission of higher amounts of FIR. For example, amethyst and tourmaline are stones that deepen the benefits of the infrared radiation to the body of a user and they also emit negative ions. Charcoal ceramic ball may function as a detoxifying agent that draws toxins out of the body of the user. Further, yellow mud ceramic ball is a balancing agent that is cooling to the infrared radiation. In one embodiment, two separate layers of yellow mud layer and charcoal layer may be bound as one. Not only do both materials intensify far-infrared radiation of system 100, both also control any smell generated during the sweating process of a user. The grid shown in FIG. 2 may be configured to hold all the stones and materials together.

Moreover, system 100 of FIG. 2 may include 10 pieces of medical magnets configured to help activate blood circulation of a user. A yellow mud charcoal bonding fabric 206 may be included to promote higher FIR generation. In one embodiment, system 100 may include multiple volatile organic compounds (VOC) free polyurethane leather (PU) layers or portions 208, 210 for water-proof, fire-proof and melt-proof purposes. An ELF proof charcoal layer 212 may be used to provide additional grounding effect, such that system 100 exhibits low to no ELF radiation or harmful frequencies to the user in the sauna blanket.

One or more sensors 214 may be placed and located inside the blanket and configured to detect various working conditions of the blanket. In one embodiment, sensors 214 may include at least one electronic temperature sensor for monitoring and detecting the internal temperature of the blanket and regulating the heating temperature accordingly. Multiple temperature levels (e.g., targeting from level 1 to 8 with 1 being the default and lowest) may be implemented for the blanket. For example, the at least one temperature sensor of sensors 214 may be in thermal communication with the heating wires 106 and measure the temperature inside the blanket. Sensors 214 may include at least one microcontroller or integrated circuit (IC) chip configured to communicate with and control other electronic components of the blanket. For example, the at least one temperature sensor may be electronically connected to the microcontroller. As a result, the electronic readings of the at least one temperature sensor may be transmitted to the microcontroller, which in turn generates and displays alerts for the user if a detected temperature exceeds a predetermined threshold value. The microcontroller may be configured to adjust the temperature of the heating wires 106 in accordance with a user selected temperature level.

One or more bimetals 216 may be used as a safety unit inside the blanket. In one embodiment, bimetals 216 may be connected to the heating wire line directly, such that they disconnect when the temperature of heating wires 106 detected by the temperature sensor exceeds 80° C. and reconnect to the heating wires 106 if the temperature detected to return to a normal range, thereby regulating the internal temperature of the blanket of system 100. In some implementations, system 100 may include silver fiber fabric and copper mesh fabric portions or layers (not shown) to additionally reduce harmful ELF radiation.

As also shown in FIG. 2, in a preferred embodiment, system 100 may include an oxford bonding layer 218 on the shell PU of the blanket, a layer 220 of compressed non-woven fabric, and a layer 222 housing an ELF proof charcoal layer and grounding lead wires (e.g., grounding lead wires 108 of FIG. 1).

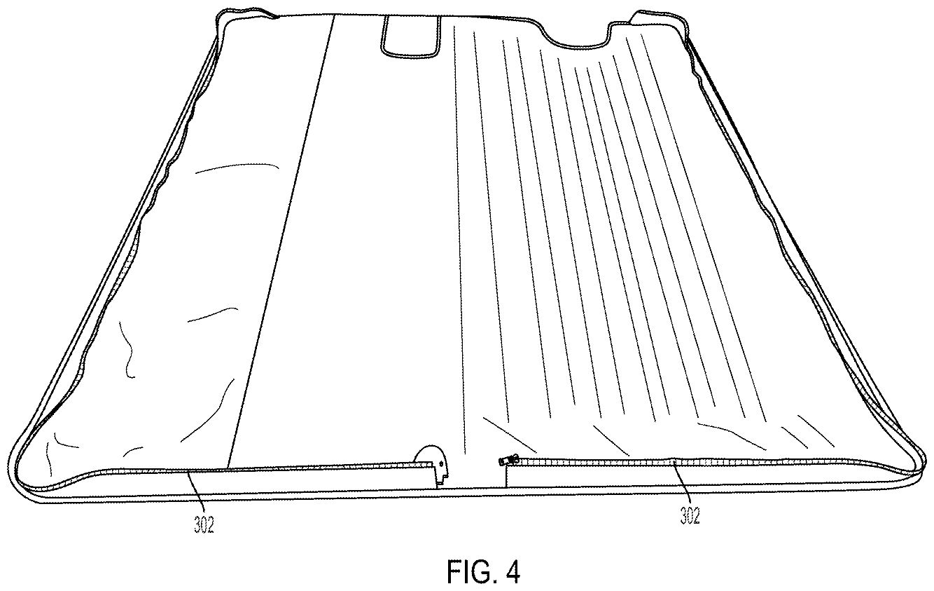

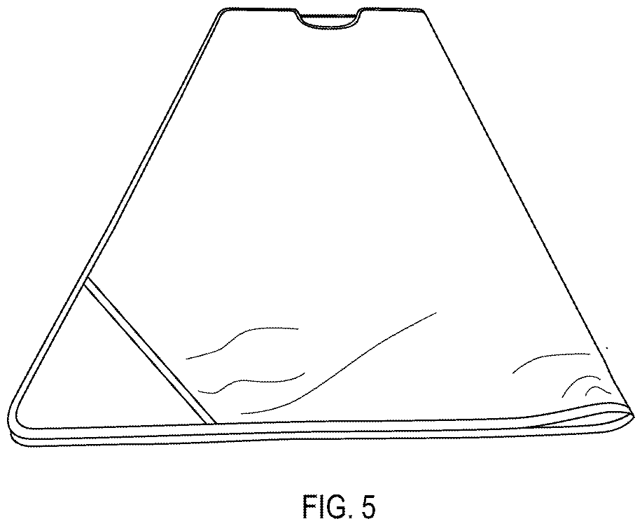

FIGS. 4 and 5 illustrate a multi-layer portable therapeutic infrared heating blanket in an open and closed form, respectively, in accordance with aspects of the present disclosure. The blanket may include a zipper closure 302 configured to open the blanket from one of the sides for easy access inside the blanket. As shown in FIG. 4, when the blanket is completely open, the bottom portion of the blanket may also be opened by detaching the two sides of the zipper 302. In one embodiment, the zipper 302 may start at one corner of the bottom of the blanket, opposite to the opening from where a user slides inside the blanket. The position of zipper 302 may be placed along the bottom and one side of the blanket, such that the user may easily close the blanket and easily open it when required. Zipper 302 may be configured to provide flexibility to the user if the user wants to use the lower portion of the blanket only. For example, the user may close zipper 302 halfway on the side of the blanket and folding the open part on the top diagonally.

The above description of the disclosure is provided to enable a person skilled in the art to make or use the disclosure. Various modifications to the disclosure will be readily apparent to those skilled in the art, and the common principles defined herein may be applied to other variations without departing from the spirit or scope of the disclosure. Further, the above description in connection with the drawings describes examples and does not represent the only examples that may be implemented or that are within the scope of the claims.

Furthermore, although elements of the described aspects and/or embodiments may be described or claimed in the singular, the plural is contemplated unless limitation to the singular is explicitly stated. Additionally, all or a portion of any aspect and/or embodiment may be utilized with all or a portion of any other aspect and/or embodiment, unless stated otherwise. Thus, the disclosure is not to be limited to the examples and designs described herein but is to be accorded the widest scope consistent with the principles and novel features disclosed herein.

1. A multi-layer portable therapeutic infrared heating system, comprising:
a heating wire layer configured to generate far-infrared radiation (FIR) when the multi-layer portable therapeutic infrared heating system is connected with an alternating current power supply;
two charcoal layers positioned on either side of the heating wire layer and configured to absorb extremely low frequency (ELF) radiation associated with the FIR;
grounding mechanism configured to transfer the ELF radiation absorbed by the two charcoal layers to a pin and ground the multi-layer portable therapeutic infrared heating system; and
a multi-layer mixture layer configured to at least additionally reduce the ELF radiation.

2. The multi-layer portable therapeutic infrared heating system of claim 1, wherein the heating wire layer includes silicone Teflon wires.

3. The multi-layer portable therapeutic infrared heating system of claim 1, wherein the system is configured to maintain a level of the ELF below 5 mV and low frequency electromagnetic field (EMF) below 2 milligauss.

4. The multi-layer portable therapeutic infrared heating system of claim 1, wherein the multi-layer mixture layer includes a selected amount of one or more of amethyst gravel, yellow mud ceramic ball, charcoal ceramic ball, white ceramic ball, and tourmaline ceramic ball, wherein the multi-layer portable therapeutic infrared heating system further comprises a grid to hold stones and materials of the multi-layer mixture layer together.

5. (canceled)

6. The multi-layer portable therapeutic infrared heating system of claim 1, further comprising a medical magnet layer including multiple medical magnets for activating a blood circulation of a user of the system.

7. The multi-layer portable therapeutic infrared heating system of claim 1, further comprising a yellow mud charcoal bonding fabric layer configured to promote a generation of the FIR.

8. The multi-layer portable therapeutic infrared heating system of claim 1, further comprising at least one charcoal layer configured to ground the multi-layer portable therapeutic infrared heating system and reduce the ELF radiation.

9. (canceled)

10. The multi-layer portable therapeutic infrared heating system of claim 1, further comprising at least one electronic temperature sensor for monitoring and detecting an internal temperature of the multi-layer portable therapeutic infrared heating system and regulating a heating temperature of the multi-layer portable therapeutic infrared heating system in accordance with a selected temperature level.

11. The multi-layer portable therapeutic infrared heating system of claim 10, further comprising a safety unit configured to disconnect the heating wire layer when the at least one electronic temperature sensor detects that the internal temperature of the multi-layer portable therapeutic infrared heating system exceeds 80° C.

12. The multi-layer portable therapeutic infrared heating system of claim 11, wherein the safety unit is configured to connect to the heating wire layer when the internal temperature of the multi-layer portable therapeutic infrared heating system detected by the at least one electronic temperature sensor returns to a normal range.

13. The multi-layer portable therapeutic infrared heating system of claim 1, further comprising silver fiber fabric and copper mesh fabric portions or layers to additionally reduce the ELF radiation.

14. The multi-layer portable therapeutic infrared heating system of claim 1, wherein the grounding mechanism comprises at least two grounding lead wires configured to connect the two charcoal layers with a grounding pin.

15. The multi-layer portable therapeutic infrared heating system of claim 1, wherein the heating wire layer includes wires made of a material selected to reduce or shield harmful electric and magnetic fields generated by the heating wire layer.

16. The multi-layer portable therapeutic infrared heating system of claim 1, wherein the multi-layer mixture layer is further configured to promote higher FIR generation, heating distribution and therapeutic benefits.

17. A multi-layer portable therapeutic infrared heating blanket, comprising:
a heating wire layer configured to generate far-infrared radiation (FIR) when the multi-layer portable therapeutic infrared heating blanket is connected with an alternating current power supply;
two charcoal layers positioned on either side of the heating wire layer and configured to absorb extremely low frequency (ELF) radiation associated with the FIR;
grounding mechanism configured to transfer the ELF radiation absorbed by the two charcoal layers to a pin and ground the multi-layer portable therapeutic infrared heating blanket; and
a multi-layer mixture layer configured to at least additionally reduce the ELF radiation.

18. The multi-layer portable therapeutic infrared heating blanket of claim 17, wherein the heating wire layer includes silicone Teflon wires.

19. The multi-layer portable therapeutic infrared heating blanket of claim 17, wherein the multi-layer portable therapeutic infrared heating blanket is configured to maintain a level of the ELF below 5 mV and low frequency electromagnetic field (EMF) below 2 milligauss.

20. The multi-layer portable therapeutic infrared heating blanket of claim 17, wherein the multi-layer mixture layer includes a selected amount of one or more of amethyst gravel, yellow mud ceramic ball, charcoal ceramic ball, white ceramic ball, and tourmaline ceramic ball, wherein the multi-layer portable therapeutic infrared heating blanket further comprises a grid to hold stones and materials of the multi-layer mixture layer together.

21. (canceled)

22. The multi-layer portable therapeutic infrared heating blanket of claim 17, further comprising a medical magnet layer including multiple medical magnets for activating a blood circulation of a user of the multi-layer portable therapeutic infrared heating blanket.

23. The multi-layer portable therapeutic infrared heating blanket of claim 17, further comprising:
a yellow mud charcoal bonding fabric layer configured to promote a generation of the FIR; and
at least one charcoal layer configured to ground the multi-layer portable therapeutic infrared heating blanket and reduce the ELF radiation.

24. (canceled)

25. (canceled)

26. The multi-layer portable therapeutic infrared heating blanket of claim 17, further comprising at least one electronic temperature sensor for monitoring and detecting an internal temperature of the multi-layer portable therapeutic infrared heating blanket and regulating a heating temperature of the multi-layer portable therapeutic infrared heating blanket in accordance with a selected temperature level.

27. The multi-layer portable therapeutic infrared heating blanket of claim 26, further comprising a safety unit configured to disconnect the heating wire layer when the at least one electronic temperature sensor detects that the internal temperature of the multi-layer portable therapeutic infrared heating blanket exceeds 80° C.

28. The multi-layer portable therapeutic infrared heating blanket of claim 27, wherein the safety unit is configured to connect to the heating wire layer when the internal temperature of the multi-layer portable therapeutic infrared heating blanket detected by the at least one electronic temperature sensor returns to a normal range.

29. The multi-layer portable therapeutic infrared heating blanket of claim 17, further comprising silver fiber fabric and copper mesh fabric portions or layers to additionally reduce the ELF radiation.

30. The multi-layer portable therapeutic infrared heating blanket of claim 17, wherein the grounding mechanism comprises at least two grounding lead wires configured to connect the two charcoal layers with a grounding pin.

31. The multi-layer portable therapeutic infrared heating blanket of claim 17, wherein the heating wire layer includes wires made of a material selected to reduce or shield harmful electric and magnetic fields generated by the heating wire layer.

32. The multi-layer portable therapeutic infrared heating blanket of claim 17, wherein the multi-layer mixture layer is further configured to promote higher FIR generation, heating distribution and therapeutic benefits.

33. The multi-layer portable therapeutic infrared heating blanket of claim 17, further comprising a zipper configured to open the multi-layer portable therapeutic infrared heating blanket from one side for easy access.

34. The multi-layer portable therapeutic infrared heating blanket of claim 33, wherein the zipper is configured to start at one corner of a bottom of the multi-layer portable therapeutic infrared heating blanket.

35. The multi-layer portable therapeutic infrared heating blanket of claim 34, wherein the zipper is configured to close a portion of the multi-layer portable therapeutic infrared heating blanket on one side at a selected position.
